(19) United States
(12) Patent Application Publication
(10) Pub. No.: US 2016/0144142 A1
(43) Pub. Date: May 26, 2016

(54) METERED DOSE RESPIRATORY TRAINING DEVICE AND SYSTEM

(71) Applicants: Jeff Baker, Orlando, FL (US); Francis Michael Siemer, Orlando, FL (US); Matthew Palyo, Orlando, FL (US); Seth Freytag, Winter Springs, FL (US); Christopher Chung, Orlando, FL (US); Kristin DeSanto, Winter Garden, FL (US); Paul van der Pol, Winter Garden, FL (US)

(72) Inventors: Jeff Baker, Orlando, FL (US); Francis Michael Siemer, Orlando, FL (US); Matthew Palyo, Orlando, FL (US); Seth Freytag, Winter Springs, FL (US); Christopher Chung, Orlando, FL (US); Kristin DeSanto, Winter Garden, FL (US); Paul van der Pol, Winter Garden, FL (US)

(21) Appl. No.: 14/551,523

(22) Filed: Nov. 24, 2014

(51) Int. Cl. A61M 15/00

(52) U.S. Cl. CPC A61M 15/0071; A61M 15/0021; A61M 2205/13; A61M 2205/3334; A61M 2205/50; A61M 2205/18

(57) ABSTRACT

In an embodiment, a respiratory inhaler training device configured to provide stepwise instructions for using the device to a user in a sequence of steps is provided. The respiratory inhaler training device includes a housing defining a channel with an inlet and an outlet, at least one actuation mechanism simulating provision of medicament, at least one fluid flow rate sensor positioned so as to detect a rate of fluid flow through the channel to determine a fluid flow rate, a signal output component for providing an output, a microprocessor and a timekeeping component, a storage medium component associated with the microprocessor comprising a database of instructions pertaining to the sequence and/or timing of steps for using the device stored thereon.

### BACKGROUND

Respiratory inhalation devices are used to treat a number of different diseases and conditions, or to relieve symptoms associated therewith including asthma, chronic obstructive pulmonary disease (COPD), cystic fibrosis, among other illnesses. Use of respiratory inhaler devices can be complex and oftentimes difficult, as each type of device and/or each medication includes its own Instructions for Use (IFU). Some medications have numerous steps which must be followed in a precise manner in order to receive an accurate dosage of the medication. Moreover, some respiratory inhalation devices and medication require precise timing of multiple steps to be performed in conjunction with one another. Without proper training, these devices can be extremely difficult to use and can create a sense of anxiety in a user.

Self-management of chronic conditions can be complex and self-medication can often result in an unpleasant and/or ineffective experience. Experiences of patients that are new to medicament delivery devices include anxiety, errors, and non-compliance. Medicaments that are administered at home with devices account for over 200,000 adverse events in the Food and Drug Administration (FDA) Adverse Event Reporting System (AERS) database. The FDA considers patient errors as device failures. Consequently, the FDA is attentive and oftentimes critical of self-management devices. Device developers therefore, focus on ease of use of the device in development, but do not focus much attention to the ease of learning, which is the most relevant and most critical factor in reducing patient errors.

Asthma affects approximately 235 million people worldwide, a number estimated to grow to 400 million by 2025, with the highest growth in children. It is the most common chronic disease among children. In the US, 8.3% of the population (25 million people) has asthma. The prevalence of asthma in females is 29% higher than in males, According to the CDC (2009, USA only), asthma caused 8.9 million doctor visits, 1.9 million emergency room visits, 479,300 hospitalizations, and 3,400 deaths.

Asthma is commonly treated through therapies consisting of maintenance (control) and quick relief (rescue) medications. Maintenance medications are preventative and when used over time, can reduce airway inflammation and risks associated with asthma. Quick relief medications are reactive medications used to treat acute symptoms and dilate constricted airways. Proper medication and lifestyle management can limit the occurrence and severity of asthmatic episodes. Asthma plans, created by patients with their HCP, typically monitor lung performance and symptoms to adjust treatments and dosing as needed.

Multi-sensory learning is very important for effectively learning new behaviors, particularly when there are multiple steps and requirements that must be met, such as with the use of self-medication devices. Additionally, it is critically important that these devices be used correctly to assure compliance and effective administration of medicaments to patents in need. Triggered by sensory stimulation, the brain constantly creates new network connections between neurons. Each time we learn, the new connections slightly change the brain. Multisensory learning is based on several neurophysiological and psychological principles, including i) the human body has approximately 20 sensory systems, the sensory stimuli most relevant to learning are auditory, visual, somatosensory (tactile), gustatory, and olfactory; ii) multisensory learning engages multiple sensory modalities, which are interpreted in distinct areas of the brain; iii) sensory stimuli are integrated in the superior colliculus, the structure of the superior colliculus, located in the midbrain, contains a high proportion of multisensory neurons; iv) the more senses are stimulated, the more network pathways are available for retrieval, thus, the better we learn. This is as long as each sense gets a signal at the same time, space and meaning; v) it is autonomous and ubiquitous, the brain is already wired for it and there are many instances of multisensory learning in everyday life; and vi) not only do the senses complement one another, they can modulate (strengthen) one another. This mutual reinforcement facilitates processing and retention in the brain.

There are numerous benefits of multisensory learning, some of which include: a) under the right conditions, information is processed and interpreted faster; b) better retention in memory and information is remembered over a longer period of time; c) distraction is avoided—if a sense (eye, ear, etc.) is not in use for learning, it will still be active, and if it receives a signal that is not in agreement with the subject matter, it all aspects of learning are interrupted; d) with multiple senses occupied, is easier to hold attention; e) a single sensory cue activates all areas of the brain that have received stimuli (cross-modal processing), this phenomenon is the most surprising and powerful discovery of the use of magnetic resonance imaging (MRI) in neuroscience, for example; and f) people who have entrenched neural pathways (older people), multisensory learning is especially helpful in the acquisition of new knowledge that is contradictory to prior experience.

To take advantage of the neurological mechanisms in the brain, certain requirements need to be considered in regard to medicament training devices. The requirements for multisensory learning are: spatially, the sources of stimuli have to be in close proximity; temporally, the sources of stimuli have to be synchronous; semantically, the stimuli have to be congruous (see above); minimize sensory redundant information (both within mode and in between mode), otherwise, a split in attention will result. Additionally, active learning induces greater multisensory integration compared to passive observation. Active motor learning, where the learner engages in the real thing, modulates the establishment and processing of multisensory connections. Functional connectivity between visual and motor cortices is stronger after active learning than passive learning.

A four stage process occurs with educating a new patient to use an unfamiliar medicament delivery device. The first stage includes the training of sales representatives of a pharmaceutical company wherein the company has extensive control over the consistency of the training message. In the second step, the sales representative trains the healthcare provider (HCP). Because both the sales representatives and healthcare providers are often stressed for time, and due to the enormous variance in training environments, message erosion can occur. The healthcare provider then trains the patient. Typically, such a training session takes 30 minutes, a significant amount of time in a healthcare provider's day, and an amount of time the healthcare provider is reluctant to give up. Because of the enormous variance in educational backgrounds and teaching experience of healthcare providers, significant message erosion is takes place in this four stage process. Lastly, the fourth step includes the patient who learns how to use the device and practices repeatedly with the device at home.

Metered dose Inhalers (MDI) are handheld devices used to provide medication to patients with different medical conditions. The MDI includes a pressurized canister of medicament, and delivers a specific amount of medication from the pressurized canister to a user in aerosol form. The MDI requires a user to press on the device while inhaling the medication into the lungs. MDIs use a chemical propellant to deliver medication from the inhaler. Timing of inhalation and manipulation of the MDI device are critical to effective use of a MDI and receipt of a proper dose of medicament.

The most common errors associated with the use of a MDI include 1) failing to shake the MDI prior to use; 2) failing to prime the MDI prior to use; 3) lack of coordination of actions and/or improper timing of actions; 4) failing to inhale for a sufficient time period; 5) failing to hold one's breath for a sufficient time period; and 6) failing to orient the MDI correctly during use. These and other errors that can occur during the use of the MDI device may cause users to receive an incorrect or incomplete dose of medicament, or destroy the MDI among other concerns.

### SUMMARY

In an embodiment, a respiratory inhaler training device configured to provide stepwise instructions for using the device to a user in a sequence of steps is provided. The respiratory inhaler training device includes a housing defining a channel with an inlet and an outlet, at least one actuation mechanism simulating provision of medicament, at least one fluid flow rate sensor positioned so as to detect a rate of fluid flow through the channel to determine a fluid flow rate, a signal output component for providing an output, a microprocessor and a timekeeping component, a storage medium component associated with the microprocessor comprising a database of instructions pertaining to the sequence and/or timing of steps for using the device stored thereon. The respiratory inhaler device includes one or more program code modules stored on the microprocessor or the storage medium component, or a combination thereof, wherein the one or more program code modules includes a first program code module for causing the microprocessor to provide a first instruction, a second program code module for causing the microprocessor to provide a subsequent instruction based on a current register, and a third program code module causes the microprocessor to compare one or more fluid flow rate values detected with one or more predetermined fluid flow rate values, wherein when the detected fluid flow rate values do not meet the predetermined fluid flow rate values, an error condition is set in the current register, and a signal output component is initiated to provide an error message to the user, wherein the one or more predetermined fluid flow rate values includes at least 30 L/min, which is typically the minimal inhalation force to carry the aerosol droplets deep into the lungs, in some non-limiting embodiments.

In another embodiment, a respiratory inhaler training system configured to provide instructions for using a respiratory inhaler training device to a user in a sequence of steps is provided. The system includes a respiratory inhaler training device including a housing defining a channel with an inlet and an outlet, the respiratory inhaler training device including an actuation mechanism, the actuation mechanism configured to simulate provision of medicament. The system including a respiratory inhaler training container, wherein the training device communicatingly connects to the respiratory inhaler training container, a signal output component associated with the container and/or the training device, said signal output component configured to provide a feedback to the user based on a use of the system, and a microprocessor associated with the respiratory inhaler training device and/or container configured so as to control a provision of the instructions to the user in the sequence of steps, wherein the respiratory inhaler training container comprises a power source.

In a further embodiment, a respiratory inhaler training system is provided. The respiratory inhaler training system includes a housing defining a channel with an inlet and outlet, the housing including two or more mechanical components movable relative to one another during use of the system, wherein movement of the two or more mechanical components relative to one another produces an audible output. The system further includes a signal output component, at least one actuation mechanism associated with the housing, the actuation mechanism simulating provision of medicament, and a signal receiving component for receiving the audible output from the signal output component.

In still a further embodiment, a respiratory inhaler training device configured to provide stepwise instructions for using the device to a user in a sequence of steps is provided. The respiratory inhaler training device includes a housing defining a channel with an inlet and an outlet, at least one actuation mechanism simulating provision of medicament, at least one fluid flow rate sensor positioned so as to detect fluid in the channel, at least one accelerometer to detect movement of the device, at least one contact sensor disposed between the housing and the actuation mechanism to detect activation and/or deactivation of the actuation mechanism, at least one orientation sensor to detect an orientation of the device, at least one signal output component for providing an output to the user, and a microprocessor comprising a timekeeping component, wherein the timekeeping component is configured to measure an elapsed time during and/or between the sequence of steps. The training device further includes a storage medium component associated with the microprocessor comprising a database of instructions pertaining to the sequence of steps for using the device stored thereon, one or more program code modules stored on the microprocessor or the storage medium component or a combination thereof, wherein the one or more program code modules include a first program code module for causing the microprocessor to provide a first instruction, and a second program code module for causing the microprocessor to provide a subsequent instruction based on a current register, wherein based on a signal received from the at least one fluid flow rate sensor, at least one accelerometer, at least one contact sensor, and/or at least one orientation sensor, and/or an input received from the user, the microprocessor detects a condition of the device.

### BRIEF DESCRIPTION OF DRAWINGS

FIG. 1 is a perspective view of an embodiment of a respiratory inhaler training device.

FIG. 2 is a perspective view of another embodiment of a respiratory inhaler training device.

FIG. 3 is a perspective view of a respiratory inhaler training system embodiment.

FIG. 4 is a perspective view of another respiratory inhaler training system embodiment.

FIGS. 5A-D are tables demonstrating signals of two sensors over time.

FIG. 6 is a table providing an embodiment of a set of registers for a training device embodiment or training system embodiment.

FIG. 7 is a flow chart providing a logic algorithm embodiment for an embodiment of a training device and/or system.

FIG. 8 is a flow chart of a logic algorithm embodiment for detecting air flow sensor input.

FIG. 9 is a flow chart of a logic algorithm embodiment of an error condition subroutine.

FIG. 10 is a flow chart of a logic algorithm embodiment of a confirmation subroutine

FIG. 11 is a table providing an embodiment of a data structure of the system and/or device.

FIGS. 12A-12B are graphical illustrations demonstrating two non-limiting examples of synchronized uses of the training device to provide a correct dose of medicament.

FIG. 13 is a flowchart of a logic algorithm embodiment of a subroutine to start and resume use of the device and/or system.

FIG. 14 is a flow chart of a logic algorithm of a language selection subroutine.

FIG. 15 is a flow chart of a logic algorithm for using the previous selection input.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

The use of a metered dose respiratory inhaler requires precise coordination of user actions to receive a proper dosage of a medicament. Practice using an inhaler can assist a patient in establishing autonomous time and/or motor skills of coordinated actions. Only frequent use of the inhalation trainer can improve timing and provide a user with the tools to develop the technique to use the inhaler medicament delivery device to receive a required dose of medicament. Benefits of the respiratory training device embodiments described herein include familiarizing patients with the training device, which closely resembles the medicament delivery device on the market so as to increase patient confidence and comfort in using the device. This will assist in reducing patient error in using the device, allow a user to develop autonomous motor skills and reduce the amount of time of and reduce the burden on health care providers to assist and train patients to use the device. Furthermore, respiratory training devices will provide benefits including reducing error rate when using the drug delivery inhaler device, as well as increase patient comfort and confidence in using the drug delivery inhaler device.

Two primary environments for training a user to use a respiratory inhaler medicament delivery device with the use of the respiratory inhaler training device includes: in a healthcare provider's office or other healthcare setting in which a physician or a nurse most likely educates themselves on how to use the respiratory inhaler training device. The healthcare provider will then use the training device to train the patient in a typical exam room setting that includes chairs and a countertop, but no table. The second setting is an at home setting, wherein the patient will practice with the training device at home, either alone or with, in most instances, a non-medically trained companion. The training device will be used, therefore, without medical supervision before the first use of the medicament delivery respiratory inhaler device. A refresher training with the training device may occur as needed just before the subsequent use of the medicament delivery respiratory inhaler device.

The Food and Drug Administration (FDA) mandates that all users of medical devices used in home healthcare have readable and understandable instructions in order to operate these devices safely and effectively. The instructions for use of various medical inhaler device describe numerous steps the patient has to take to safely administer a full dose of medication using the device. It is important for the patient to read the IFU in addition to using the training device to assist the user in learning to correctly use and increase comfort in using the medicament delivery device. The sequence of steps in the instructions for using the device are critically important, therefore, the training device follows the instructions for use sequence.

Embodiments of a training device provided herein for use as a respiratory inhaler training device provide an ability to identify mistakes in the use of a respiratory inhaler delivery device before the medicament delivery device is used by a patient, increase compliance in proper use of the medicament delivery device, improve adequacy of use of the medicament delivery device, identify errors patients make with the device, intervene where a patient makes a mistake, and guide the patient through proper use of the device. Error recognition may occur through the use of sensors, including fluid flow rate and fluid direction sensors, orientation sensors, contact sensors, accelerometers, among other types of sensors that may be used. The device is able to teach a user the coordination required for proper medicament administration via a respiratory inhaler device. The device tracks the proper sequence of actions and precise timing of these actions and provides auditory and/or visual feedback to a user accordingly. The training experience allows a patient to establish muscle memory. Some of the tracked user actions include: 1) inhalation, wherein a fluid flow rate sensor is used to detect sufficient inhalation force, 2) activating the actuation mechanism, wherein a contact sensor is used, 3) measuring elapsed time, wherein a timekeeping component of the microprocessor is used, 4) providing a signal to prompt a user when to perform an action (i.e., activating the actuation mechanism), wherein an audio output is used such as a beeping sound, and 5) measuring inhalation volume, wherein the fluid flow sensor rate and timer are used.

### DEFINITIONS

A “predetermined value” as used herein, for example, includes but is not limited to a value or range of values relating to an event involving use or operation of the device. These may include, but are not limited to thresholds, ceilings, baselines or range values that are desired or undesired for a particular event. Examples of predetermined values include, but are not limited to, a predetermined orientation value, predetermined time value, or a predetermined contact value, in addition to other predetermined values described herein refers to a value that is used as a reference value in relation to a value, signal, or indication that is detected by, for example, a sensor of the delivery training device. Predetermined value may include an optimal value, or a sub-optimal value, or any value there between.

In one example, a predetermined orientation value may include a 90 degree angle between the device and a target region for the device, an additional predetermined orientation value may include a 10 degree angle between the device and a target region for the device. At either predetermined orientation value, or at any value there between, a signal output component may be initiated. The signal output component may therefore be an error message or a congratulatory message, for example.

The term “condition” as used herein includes but is not limited to a user input, a status of the device, anything that is sensed by the device, correct or incorrect stepwise activities, usage of the device over time, among other conditions. A condition may be detected based on one or more values received from the device or a sensor of the device.

The term “error condition” as used herein includes but is not limited to a condition pertaining to a mistake by the user in using the device, whether the mistake is incorrect positioning or contact between the device and the user, or whether the mistake is an out of order step, a step that exceeds or fails to meet predetermined time value (such as an undue pause during or between steps, or insufficient time for conducting a step or transition between steps). Error conditions may also include errors of the device itself, including low or lack of power or failure to operate as intended.

The term “timekeeping component” as used herein includes, but is not limited to, a component of the microprocessor for keeping time such as, for example, a clock or a timer. The timekeeping component, in one non-limiting embodiment may be used to keep time between inhalations with the device, to keep time regarding a duration of an inhalation, or to identify an amount of time between uses of the device, for example.

The term “fluid” as used herein to refer to, for example a fluid flow rate sensor, includes but is not limited to air, liquid, gas, powder, or any other such substance as known in the art to be included by the term fluid. The terms “air flow rate sensor,” “air flow sensor,” “fluid flow rate sensor,” and “fluid flow sensor” are used herein interchangeably to refer to a sensor that can detect any type of fluid, including, but not limited to air.

The term “signal output component” as used herein includes, but is not limited to, a component which may provide an audible, visual, gustatory, olfactory, or tactile output. It includes speakers which provide audio output, mechanical components of a device which move with or against one another to produce either tactile output, visual output, or audio output (i.e., mechanical clicks) or a combination thereof. The signal output component may include one or more lights, displays, or videos. Multiple signal output components may be provided in or associated with one device or one system. Various signal output components can be used in conjunction with one another. Certain signal output components may provide multiple sensory stimulation or signals, such as a video tutorial providing instructions for use which provides both visual and audio feedback, stimulation and instruction to a user, for example. The signal output component can be provided for the benefit of a user to observe or to indicate information to the user of the system or device. The signal output component may also be detectable by a remote or external device, for example. In some non-limiting embodiments, the signal output components may produce a whistle or sound made as a result of inhalation through the respiratory training inhaler device by a user, for example. Therefore, the signal output component may refer to a particular orientation of the parts of the device such that inhalation through the device produces an audible output or parts of the device that move relative to one another to provide an output which can be received and/or analyzed either by a user or by a remote or external device, in non-limiting examples. This output, as aforementioned, may be visual, auditory, tactile, gustatory, olfactory, or any combination thereof. In some non-limiting examples, the output may include a light, a radio signal produced by a signal output component such as an emitter, or a vibratory output produced by a signal output component. The term “associated” or “association”, as used herein, includes but is not limited to direct and indirect attachment, adjacent to, in contact with, partially or fully attached to, and/or in close proximity therewith. The term “in conjunction with” as used herein includes but is not limited to synchronously or near synchronous timing, the phrase may also include the timing of outputs, where one output directly follows another output.

The term “value” as used herein, may refer to a specific value or a range of values.

The term “fluid flow rate” as used herein includes a rate of fluid movement through a forced inhalation or exhalation of a user over time as measured by a fluid flow sensor, in non-limiting embodiments. Fluid flow rate is measured as volume of fluid movement over time. In order to receive a proper dose of some medications, there is a required fluid flow rate over a specified time period that one must achieve while using a respiratory inhaler device.

In regard to a metered dose inhaler medicament delivery device, a fluid flow rate of at least approximately 30 L/min or more is recommended to receive the medicament deep into the lungs for correct administration of medicament. A volume of air required to create sufficient speed to carry a plume of droplets deep into the lungs when using a metered dose inhaler device, in non-limiting embodiments may be approximately 2 liters.

The term “fluid” as used herein includes, but is not limited to liquid, gas, or solid material, or any combination thereof.

In one embodiment, a respiratory inhaler training device to provide stepwise instructions for using the device to a user in a sequence of steps is provided. In some non-limiting embodiments described herein, there may be a predetermined time frame within which certain events or user actions should occur. For example, in one embodiment, the user should inhale for a period of time prior to activation of the actuation mechanism. The time period before inhalation begins is T₀. T₁ includes the time period between the beginning of an inhalation and activation of the actuation mechanism, and T₂ includes a time period between activation of the actuation mechanism and the end of the inhalation. In one particular embodiment, when T₁ includes a time period between approximately 0-2 seconds, a confirmation message or congratulatory message may be output to the user. In another embodiment, when T₁ includes a time period longer than approximately 2 seconds, an error condition may occur, and an error message may be output to the user, for example.

The respiratory inhaler training device includes a housing defining a channel with an inlet and an outlet, at least one actuation mechanism simulating provision of medicament, at least one fluid flow rate sensor positioned so as to detect fluid flow in the channel, a signal output component for providing an output to the user, a microprocessor comprising a timekeeping component, a storage medium component associated with the microprocessor comprising a database of instructions pertaining to the sequence and/or timing of steps for using the device stored thereon, one or more program code modules stored on the microprocessor or the storage medium component, or a combination thereof, wherein the one or more program code modules include a first program code module for causing the microprocessor to provide a first instruction, and a second program code module for causing the microprocessor to provide a subsequent instruction based on a current register.

In an embodiment, the respiratory inhaler training device may include multiple sensors to detect conditions of the device. The sensor may be disposed on or in the device or otherwise associated with the device. In one embodiment, the sensors may include a fluid flow rate sensor, a pressure sensor, an orientation or perpendicularity sensor, a contact sensor, a temperature sensor, or other type of sensor known to those of skill in the art to detect various elements with the device herein. In one particular embodiment, a fluid flow rate sensor may be provided in the channel of the device at or near the outlet of the device in some non-limiting embodiments. The fluid flow rate sensor may include a differential pressure sensor, a turbine (vane) sensor, or a thermal mass flow sensor, or a combination thereof, in non-limiting embodiments. In further non-limiting embodiments, the fluid flow rate sensor may include hot wire, leaf, ultrasonic Doppler or coriolis.

The signal output component of the device may provide information or output to a user based on a condition of the device, an error condition (i.e., error message) of the device, a confirmation message or congratulatory message of the device, a message in response to an input initiated by the user of the device or any interruption of the device either by the user or generated by the device itself (i.e., by a signal received from a sensor, for example) in response to use of the device by the user in a non-limiting embodiment. In a further embodiment, an output of the device from the signal output component is initiated in response to a predetermined value detected for a condition.

The medicament device may further include at least one responsive member that is reactive to user input. The responsive member may include a button, either virtual or non-virtual, a switch, a touch sensor, a toggle, a heat or tactilely sensitive response sensor, or any combination thereof, or any other such device as known in the art. The responsive member may be part of the control interface of the device. Alternatively, or in addition to being disposed on the device, at least one responsive member can be in association with the device. The control interface can be used for generating user commands, and the microprocessor is in communication with the control interface. The microprocessor may be configured and arranged to receive input from the user via the control interface, wherein the processor-based circuit includes an audio signal processor configured and arranged to provide audio to the user to instruct the user while using the medicament device during the medicament delivery or simulation/training, wherein the audio is controlled by the responsive member on the control interface via user input.

In one embodiment the sensor is an orientation sensor, the orientation sensor can detect the angle at which the device is held relative to another object (i.e., the user). An orientation sensor is typically implemented as a multi-axis MEMS gyroscope that measures inertia or angular rate, in some embodiments.

Consequently, an orientation sensor may be provided in association with the training device to detect an orientation of the device, to determine the device's position, or determine its orientation relative to a target area. In a further embodiment, a signal output component may be initiated if the detected orientation of the device meets a predetermined orientation. Certain medications may require certain modes of delivery or application, and may dictate the orientation of the device during delivery. The orientation sensor is useful in identifying the proper orientation for the device based on the medicament being administrated or the type of delivery device. For example, with respiratory inhaler devices and training devices, an orientation sensor can detect the angle at which the device is positioned. An orientation sensor may include but is not limited to a multi-axis MEMS gyroscope, in one embodiment, that measures inertia or angular rate. In one embodiment, an orientation sensor can detect if the device is held upright, which is particularly important in a respiratory device in order to receive an accurate dose.

Accelerometers may be used in the device herein to detect and measure g-forces, for example, in order to detect motion. An accelerometer may be used herein to detect whether the respiratory training device has been properly shaken prior to or during use, as required before using a metered dose inhaler.

In a further embodiment, the sensor may include a contact sensor provided to detect a contact between the device and the user. In still a further embodiment, the signal output component may be initiated if the detected contact of the device meets a predetermined contact value. The contact sensor may be provided to detect a full or partial contact between the respiratory training device and the user, wherein the signal output component may be initiated if the contact of the respiratory training device meets a predetermined contact value, or in other instances if the contact of the respiratory training device fails to meet the predetermined contact value. For example, a user may be alerted when there is no contact between the device and the user, when there is partial contact between the device and the user or when there is full contact between the device and the user. The contact may refer to contact between the user and a particular portion of the device, for example, the mouth portion of the device in a non-limiting embodiment.

The predetermined contact value may be set at 100% contact between the respiratory training device and the portion of the body of the user being used for the delivery of the medicament (i.e., the mouth), or the contact value may be set between 90-99%, or 80-88% contact such that a user can be made aware when there is sufficient contact between the respiratory training device and the user for adequate positioning for training with the respiratory training device or adequate delivery of medicament from the respiratory training device, in non-limiting embodiments. Additionally, or alternatively, in some circumstances contact sensors may be provided on the portion of the respiratory training device which is intended to contact the surface of the user where training for delivery of the medicament is to occur, therefore the contact sensor can alert the user when sufficient contact has been made. The user can also be alerted by an output from the signal output component when sufficient contact has not been made with the surface of the user via the contact sensor.

Furthermore, one or more contact sensors may be used detect whether the actuation mechanism on the respiratory training device has been activated (i.e. if contact has been made between an actuation member and an actuation member receiving portion of the device to indicate that the actuation member has been compressed so as to activate the device, in one embodiment). An output from the signal output component may be provided to the user when the contact sensor has been activated, or in other instances an output from the signal output component may be provided to the user when the contact sensor has not been activated. Activation of the actuation mechanism may initiate delivery of a dose of medicament from the device in a non-limiting embodiment.

Regarding fluid flow rate sensors, the fluid flow rate sensor may detect movement of air through the respiratory training device. The fluid flow rate sensor may sense the volume of air through the outlet in a given time. A fluid flow rate sensor can indicate when the patient inhales with sufficient force. This helps the patient establish muscle memory around the lungs. As such, it is one of the most important sensors to train a patient to use a metered dose inhaler. Fluid flow rate sensors can be implemented using technologies including: a differential pressure sensor (strain gauge) using the Bernoulli principle, and a thermal mass flow sensor, using the thermal transfer principle. During use of the inhaler training device, the fluid flow senor can detect inhalation before and after the actuation mechanism is activated. The actuation mechanism may include a button, in one embodiment, which may be depressed or otherwise actuated to activate the device. In a metered dose inhaler, activation of the actuation mechanism releases medicament to a user.

The fluid flow rate sensor can be used to sense a volume of air as the air moves through the mouth piece portion of the device in a given time, for example. Therefore, the fluid flow rate sensor can be used to detect when a user inhales with sufficient force (i.e., the force that would be required to deliver a full dose of the medicament in a medicament-containing medicament delivery respiratory inhaler). Notifying a user when the volume of air inhaled and/or timing of the inhalation is correct and/or incorrect can provide critical training to the user. In one non-limiting embodiment, a pressure or turbine sensor may be used to detect fluid flow rate. In other embodiments, a thermal mass flow sensor or differential pressure sensor may be used.

The respiratory training device described herein can provide training to a user for any type of respiratory inhaler or other respiratory device. While the metered dose inhaler device is specifically focused on herein, the embodiments provided herein are not intended to be limited to use for training for these devices only, and may be used to train for using other types of respiratory devices. The inhaler training device may further include a responsive member to allow a user to select which medicament delivery device the user would like to train him or herself to use with the aid of the respiratory inhaler training device and/or system. This selection may be made by any means known in the art, including but not limited to, by an input of a particular code into the respiratory inhaler training device and/or system which pertains to a specific medicament or medicament delivery device, a barcode scanner associated with the training device used to scan a bar code on the medicament delivery device and/or packaging therefore, for example, or alternatively, in another non-limiting example, the respiratory inhaler training device and/or system may be pre-programmed or use to train a user to use only one or more specific type(s) of respiratory inhaler medicament delivery devices.

Audible instructions are typically easier to follow than written instructions. Audible instructions can also be processed while a user is handling the training device. As a result, the brain interprets the instructions faster, retains them longer, and retrieves them easier. Each step in the IFU has its own script, and each script is a separate audio file, in one embodiment. An embodiment of the respiratory inhaler training device may have multiple sets of files, each in a different language. The audio technology in an embodiment of the respiratory inhaler training device may include the following characteristics: the bit depth of the audio chip is 16-bit, the audio sampling rate is 16 kHz, maximum bit rate (hardware) is 256 kbps, and available memory size for audio storage is 32 MB, in one particular non-limiting embodiment.

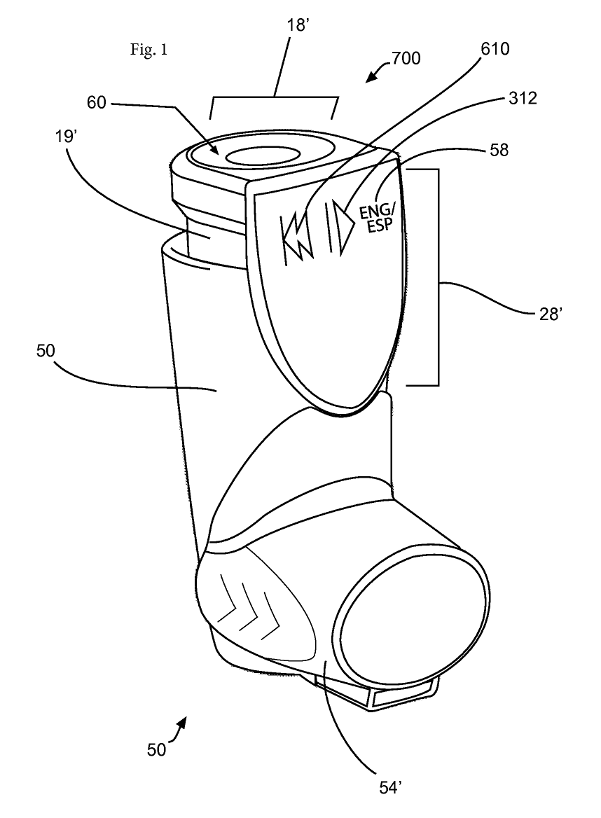

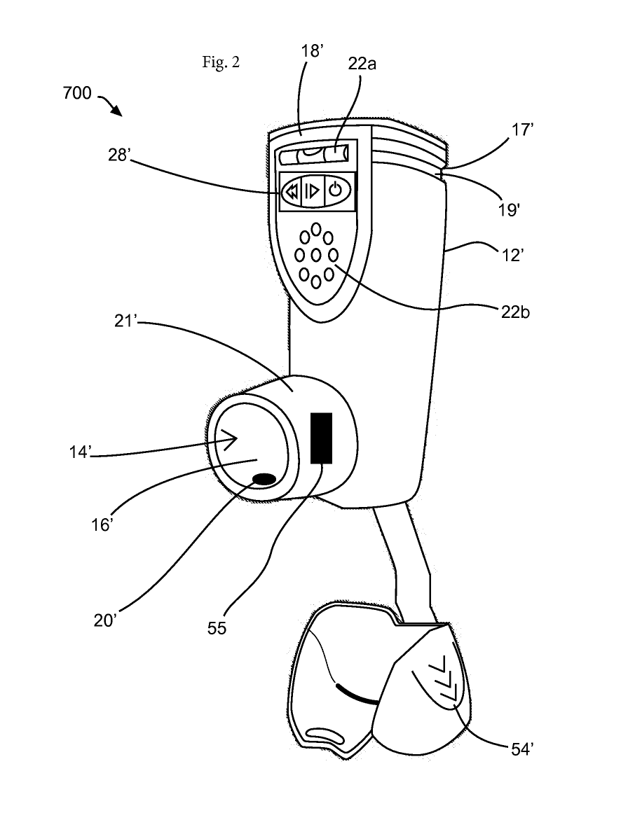

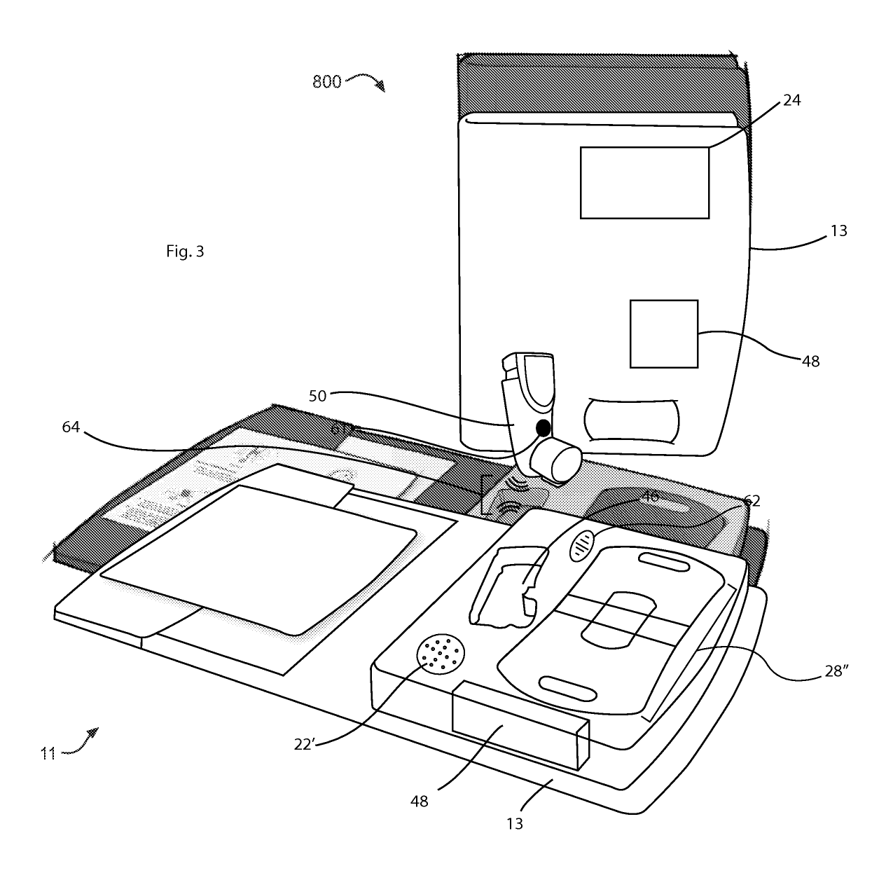

Turning to the Figures, FIG. 1 shows a perspective view of an embodiment 700 of a respiratory inhaler training device 50 having a housing 12′, an inlet 17′, an outlet 16′ (shown in FIG. 2), and a fluid flow channel disposed within the housing 14′ (shown in FIG. 2). A training canister 19′ is provided in FIG. 1 as well as a training device cap 54′. A control interface 28′ is provided on a portion of the housing 12′, wherein the control interface 28′ includes responsive members reactive to user input including a previous button 610, a start/pause button 312, and a language button 58, in one embodiment. The device 50 further includes a counter 60, providing a visual indication of time to the user during the training with the training device 50.

FIG. 2 provides another perspective view of an embodiment 700 of the respiratory inhaler training device 50, wherein the cap 54′ is removed from the mouthpiece 21′ of the training device 50 exposing the outlet 16′, a portion of the channel 14′, and sensors 55, 20′. Sensor 55 is provided to detect removal or placement of the cap 54′ on the mouthpiece 21′, and may also be configured to detect contact or proximity of the user on the mouthpiece 21′. Sensor 20′ is a fluid flow rate sensor, the purpose of which will be describe below. There may be more than one sensor provided to achieve these effects. The sensors may include contact sensors, proximity sensors, or other types of sensors known to those skilled in the art to do the same. Signal output components 22 are provided on the housing 12′ of the training device 50. One signal output component may include a visual indicator of time, which may include a display, a light, a screen, or other type of visual indication in one embodiment. A signal output component 22a may include a level as shown in the Figure herein or other component to indicate proper alignment, positioning, or orientation of the device during use. Another signal output component 22b is provided in FIG. 2 on the housing 12′, wherein the signal output component 22b includes an audio output, particularly a speaker, in a non-limiting embodiment as shown in herein.

In an embodiment of the respiratory inhaler training device as provided in FIG. 3, a respiratory inhaler training system 11 configured to provide instructions for using a respiratory inhaler training device 10′ to a user in a sequence of steps is provided. The system 11 includes a respiratory inhaler training device 50, (although any of the other embodiments of training devices may be used with the system 11, including training device embodiments 10, 10′), a respiratory inhaler training container 13, wherein the respiratory inhaler training device 50 communicatingly connects to the respiratory inhaler training container 13, a signal output component 22′ associated with the respiratory inhaler training container 13, a microprocessor 24 associated with the respiratory inhaler training device 50 or container 13 (shown as disposed within the container 19 in FIG. 3) configured so as to control a provision of the instructions to the user in the sequence of steps. Disposing components of the system 11 within the container 19 allows the device 50 to be smaller. A training device compartment 46 may be provided in the container 19 in non-limiting embodiments. Signal output components 22 may also be provided in either of the device 50 or the container 19 of the system 11. In the embodiment 800 of the system 11 shown in FIG. 3, a speaker 22′ is provided as a signal output component, through which audible output can be provided to the user, such as feedback during use of the system 11 or instructions for use of the system 11, in non-limiting embodiments.

In the embodiment 800 of the system 11, the respiratory inhaler training container 13 includes a power source 48. The respiratory inhaler training container 13 communicatingly connects to the respiratory inhaler training device 50 with a wired and/or a wireless connection 64 (as shown in FIG. 3). In a further embodiment, the respiratory inhaler training device 50 is powered by the container 13. The wireless connection may include Bluetooth® technology and/or Radio-Frequency Identification technology (RFID), in non-limiting examples, to provide communication of information there between. Therefore, in a non-limiting embodiment, the container 13 may include an RFID transponder 62 and the respiratory inhaler training device 50 may include an RFID tag 61, whereby the RFID tag 61 is energized and powered by the RFID transponder 62 in the container 13. In an alternative embodiment, the power source 48 is in the respiratory inhaler training device 50 or in both the respiratory inhaler training device 50 and the container 13.

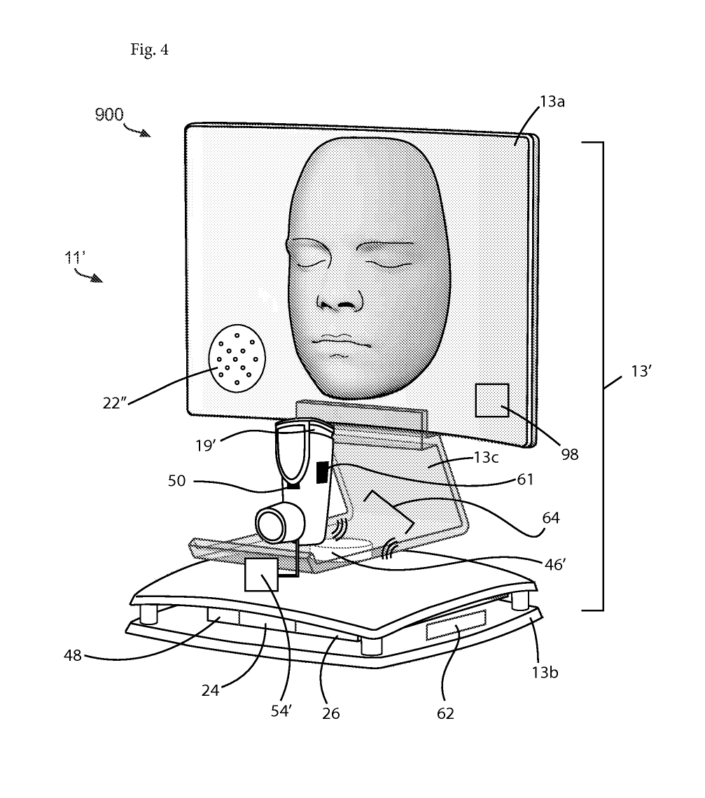

In another embodiment 900, as shown in FIG. 4, a respiratory inhaler training system 11′ configured to provide instructions for using a respiratory inhaler training device 50 to a user in a sequence of steps is provided. The system 11′ includes a respiratory inhaler training device 50, a respiratory inhaler training container 13′, wherein the respiratory inhaler training device 50 communicatingly connects to the respiratory inhaler training container 13′, a signal output component 22″ associated with the respiratory inhaler training container 13, a microprocessor 24 associated with the respiratory inhaler training device 50 or container 13′ (shown as within the container 13′ in FIG. 4), and configured so as to control a provision of the instructions to the user in the sequence of steps. The instructions may be stored as memory on a storage medium component 26, in one embodiment. Moreover, the device 50 and the container 13′ may communicatingly connect by wired and/or wireless connection 64 as described in reference to FIG. 4 above. The wireless connection may include Bluetooth® technology and/or Radio-Frequency Identification technology (RFID), or both. The container 13′ may include the RFID transponder 62 and the training device 50 may include an RFID tag 61. The upper portion of the container 13′ provided in FIG. 4 may include a display 13a, which in FIG. 4 resembles a human face, for example, and may be formed such that a user can practice using the training device 50 with the face on the display 13a of the container 13′.

A lower portion of the container 13′ includes a base 13b and a stand 13c, wherein the microprocessor 24, storage medium component 26, power supply (not shown in FIG. 4) and other components of the system may be stored therein. The base 13b and the stand 13c provide support for the display 13a of the container 13′. The display 13a may include one or more lights, LED display, picture, video display with or without audio, the display may provide a mirror for the user to watch him or herself while using the training device 10′ in non-limiting embodiments. The display 13a may include a how-to video that follows the IFU for the medicament delivery device and in some embodiments it may provide a clock or a timer for the user.

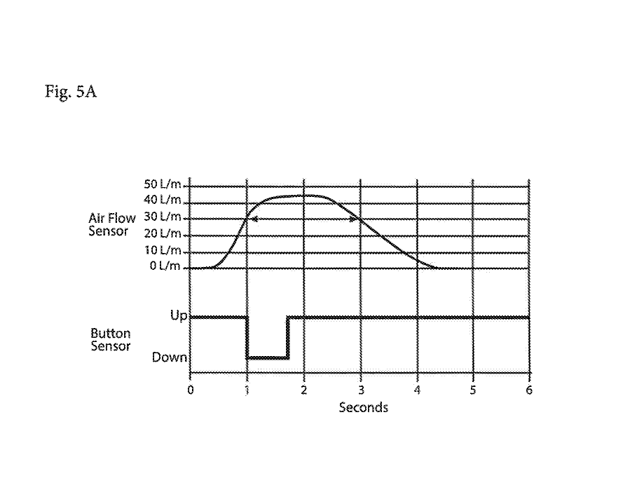

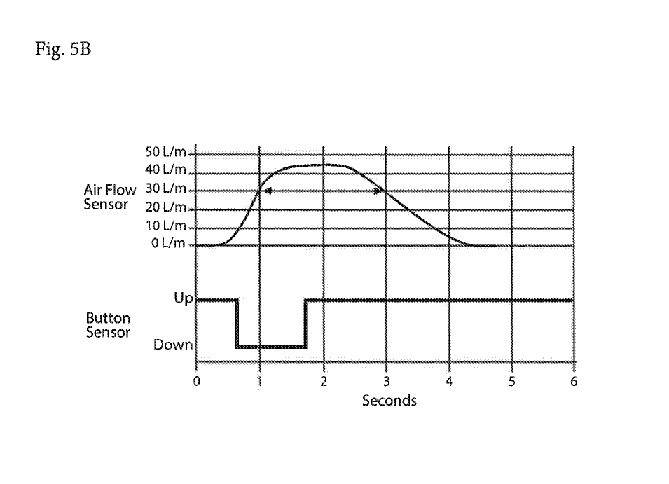

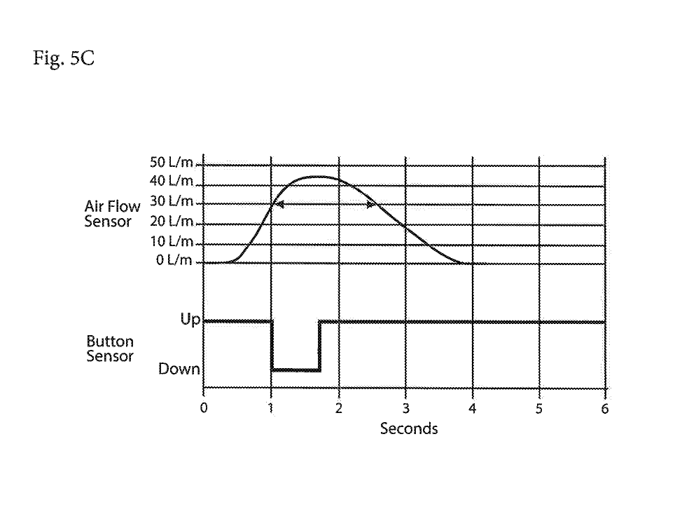

A control interface comprising one or more responsive members may be provide on the display 13a portion of the container 13′, in an embodiment, wherein a user may make selections via the display 13a. In another non-limiting embodiment, the display 13a may include advertisement materials for a particular location or medicament manufacturer or medicament delivery device. In another embodiment, the display 13a may be decorative. Certain conditions must be satisfied in use of the training device to prevent an error condition from occurring. Some of the most common error conditions that may occur are provided in greater detail below. In order to prevent an error condition from occurring, certain steps must be followed correctly and in a particular order. In a metered dose inhaler medicament delivery device, timing and the coordination of user actions is crucial. Consequently, the respiratory training device, when used to train a user to use a metered dose inhaler medicament delivery device, is provided to detect and correct the most common errors users make when using this device. In FIG. 5A, a table is provided which demonstrates the signals of two sensors over time. Measurements from a fluid flow rate sensor (e.g., airflow sensor) and a contact sensor disposed between the actuation mechanism and the housing (e.g., actuation mechanism sensor) are shown. The airflow sensor measures the volumetric flow of the inhalation. At approximately 30 L/m the flow rate is sufficient to carry the plume droplets of medicament into the air, in a non-limiting embodiment. The actuation mechanism sensor may only have two states in one embodiment, up (default) or down. In order to yield a full dose from this inhaler, the correct coordination of events or user actions must occur (from the perspective of the sensors), which requires at least three conditions: the inhalation is at least approximately 30 L/m, in one embodiment, the actuation mechanism is activated at, near, or after the moment the inhalation reaches approximately 30 L/m, and the inhalation is above 30 L/m for at least two seconds after the actuation mechanism is activated. Consequently, the conditions as shown in FIG. 5A would yield a full dose of medicament with a metered dose inhaler medicament delivery device.

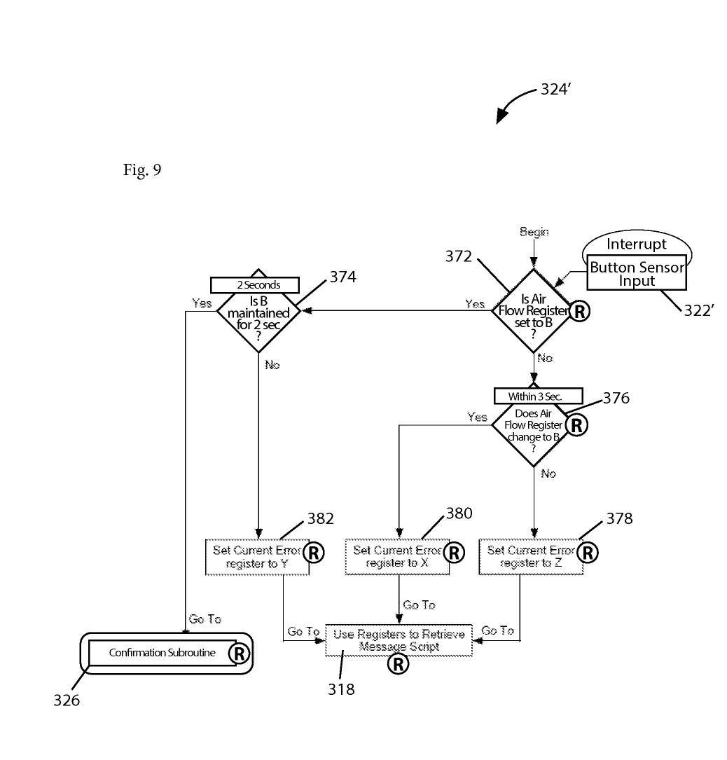

While there are many error conditions that can be identified and corrected with the training device as explained herein, the most common errors that occur with a metered dose inhaler medicament delivery device will be explained in more detail herein. The respiratory inhaler training device identifies and corrects these errors if they occur during use. One such error condition that often occurs includes premature activation of the actuation mechanism as shown in FIG. 5B. The actuation mechanism must be activated once the inhalation force (fluid flow rate during inhalation) reaches at least 30 L/m, in one embodiment. If the actuation mechanism is activated prematurely, an error condition occurs. When this error occurs during the use of the respiratory inhaler training device, the current error register is set to X in the training device (see FIG. 9). Additional information regarding the current error register will be provided herein.

A second common error that occurs in the use of a metered dose inhaler device includes an error condition in which the user fails to inhale for a certain period of time as shown in FIG. 5C. FIG. 5C shows a user compressed the button once inhalation reached 30 L/m, in a non-limiting embodiment, however the user only maintained the inhalation above 30 L/m for 1.5 seconds, which is insufficient and would not result in a full dose of medicament, in one non-limiting embodiment. If this error condition occurs (i.e., the user inhales for less than the required elapsed time calculated based on fluid flow rate), the current error register is set to Y (see FIG. 9).

While 30 L/m is used as a lower limit required for a fluid flow rate in the embodiments discussed herein, in other embodiments, this lower limit value may vary as this number is used only as an example and is not intended to be limiting. In one embodiment, the device is provided having one or more predetermined fluid flow rate values which include a lower limit and/or an upper limit. An upper limit may be provided in some non-limiting embodiments and the upper limit value may also vary. The lower limit or threshold may include 30 L/m, in one particular non-limiting embodiment. In one embodiment, a first error message may be provided if a detected fluid flow rate value is below the lower limit and/or a second error message may be provided if a detected fluid flow rate value over total time is below the predetermined value required for a dose. The first error message may be different from the second error message.

A third common error that occurs when using a metered dose inhaler device includes an inhalation force that is less than what is required to receive a correct dose of medicament (i.e., the fluid flow rate is too low) as shown in FIG. 5D. If the fluid flow rate is less than approximately 30 L/m the flow of air from the device to the user will not be sufficient to carry the plume of droplets deeply into the lungs of the user. If this error condition occurs, the current error register is set to Z (see FIG. 9).

In reference to the respiratory training device for use to train a user for using a metered dose inhaler device, three different types of messages are generally provided to the user. These messages include regular instructions, error messages, and confirmation messages. Each step in the sequence has its own instruction. The instruction is played before the user is expected to execute the step. If the user never makes a mistake, the user will only hear regular instructions in a predetermined sequence. There are eight regular instruction scripts in one non-limiting embodiment. If the user makes a mistake during the execution of a step, the user will get an error message after the step is complete or during completion of the step. The message explains what error was made and how to correct it, in one embodiment. Following the error message, the logic will take the user to a specific step, based on the nature of the error, in one example. As there are three error conditions (in one non-limiting embodiment of the device), there may be three different error message scripts, for example.

In one embodiment, there may be two confirmation message scripts: If the patient activates the actuation mechanism after the fluid flow rate is above 30 L/min, and the inhalation stays at 30 L/min for at least approximately 2 seconds after the actuation mechanism is activated, then the device lets the user know that the inhalation was good, if the patient makes a mistake in a step and subsequently executes the same step correctly, the device provides positive reinforcement, in one embodiment. Following the execution of the step, the device may play a confirmation message to confirm that the step was successfully corrected.

Embodiments of the respiratory inhaler training device and system as described herein may include a cap associated with a portion of the housing. These embodiments may further include a sensor which can provide a signal based on whether the closure member of the housing has been removed from the device, such as is required, for example, in the metered dose inhaler medicament delivery device to ensure the correct sequence of events during use of the device. If the closure member is not removed before a user activates the actuation mechanism, the user will not receive the medicament contained therein. Therefore, the housing of the respiratory inhaler training device and/or system may include at least one sensor which can detect removal of the closure member from the housing. The device and/or system can detect a condition of the device and/or system based on a signal received from the at least one sensor. Therefore, an error condition may occur if the closure member is removed at an incorrect time in the training sequence, for example, if the closure member has been removed after the device has been primed. At least one sensor may indicate if the closure member has not been properly replaced over the required portion of the housing after use of the device. The at least one sensor may include a contact sensor or a proximity sensor in non-limiting embodiments.

In another embodiment of the device and/or system herein, the device and/or system may detect if and when the device has been primed before use, and wherein the priming takes place out of order or not in conjunction with instructions as provided to the user, an error condition may result. The device and/or system may include a sensor, for example, a fluid flow rate sensor, which may detect movement of fluid within the channel of the device to determine if the device has been primed before use. Priming may consist of one or more activations of the actuation mechanism (i.e., one or more presses of the actuation button, in an embodiment) to assure that the inhaler is ready to use and will dispense the correct amount of medication. In a metered dose inhaler, the device may need to be primed before the medicament is administered to a user. Consequently the respiratory inhaler training device may include a training canister which may disperse a non-medicament spray or aerosol so that a user can experience the priming of the canister during the training. The training device may alternatively require the activation of the actuation mechanism in conjunction with instructions to prime the device and this priming may be required to occur within a certain time period once the device is powered on such that the training device or system herein can determine an out-of order sequence of events if the device is not primed before the subsequent action in the stepwise instructions has occurred (or not primed within the predetermined time period required).

In a further embodiment, the device and/or the system may detect whether the outlet portion of the housing of the respiratory inhaler training device has been placed within a user's mouth. This may be accomplished, in non-limiting embodiments, with at least one sensor, wherein the sensor may include a contact sensor associated with the output portion of the housing, a fluid flow rate sensor to detect fluid flow at or near the output, a temperature sensor, or a proximity sensor located at or near the output of the housing, in non-limiting embodiments.

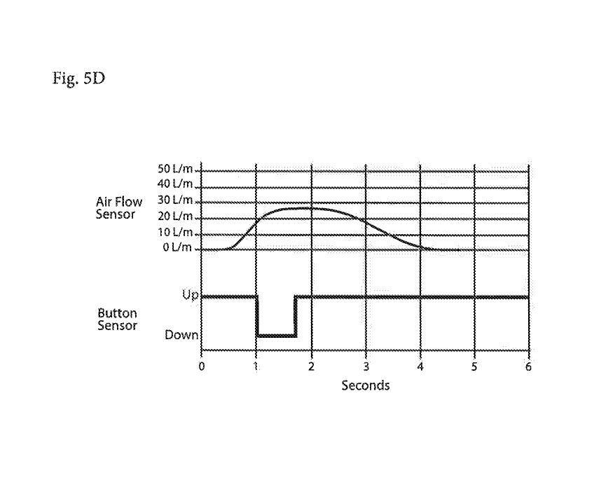

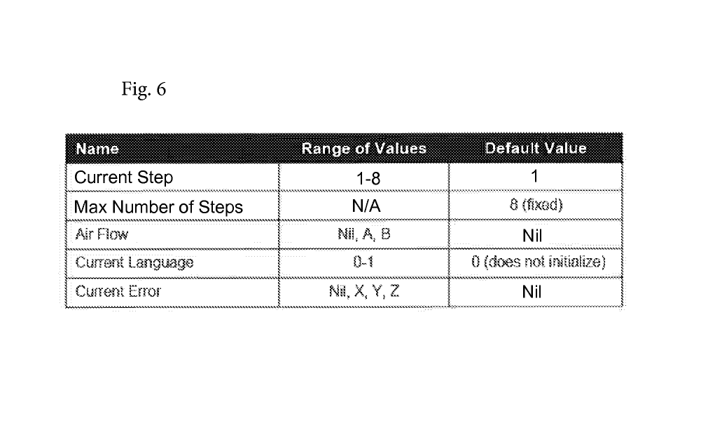

Registers include temporary values that the algorithm described herein uses to make decisions. The core logic and the subroutines can change the register values. After the user presses the START button, in an embodiment the register values are set to an initial, default value. This can also occur once the power ON or OFF button has been pressed or based on a timer in other non-limiting embodiments. The registers may be stored in non-volatile memory. FIG. 6 provides an example of a set of registers for a training device (the example provided is for a device which trains a user to use a metered dose inhaler device). The current step register keeps track of the current step. After the user presses the START button, in one embodiment, it is set to 1. If there is no error condition, this value may move to the next in a sequence at the end of each cycle.

The maximum number of steps register includes the total number of steps per round of medicament delivery for the device, in one embodiment. Once this register is set, it will not need to be changed, the pre-setting of the register may occur during manufacturing. At the end of each step, the algorithm may compare the “current step” register to the “max number of steps” register. If the “current step” value is larger, the program may terminate. FIG. 6 provides a list of registers for the training device when used as a metered dose inhaler trainer.

Based on fluid flow rate sensor output, there are three states of the fluid flow (or air flow) register: no flow (0 L/min): Nil, flow rate between 0 L/min and 30 L/min: A, flow rate above 30 L/min: B.

The current language register represents the language of the spoken instructions. If the user pushes the “language” button, the value toggles between its two options (either 0 or 1). The “current language” register does not change if the system is turned OFF. If the user pushes the START button, this register keeps the value of the last session, in one embodiment, and it can only be changed with the language button. The control interface or responsive member of the device may provide the user with the ability to change the language of the audio output of the device. Languages that the audio output may be communicated to a user include but are not limited to, English, Spanish, French, Arabic, Portuguese, Russian, Chinese, and Japanese. It is known by those of skill in the art that any language may be provided via the audio output of the device.

If the user makes a mistake, the error subroutine sets the value of the current error register. If there is no error (default), this register is set to Nil. Since there are three error conditions, in some embodiments as described herein, there are three different values (X, Y, and Z).

The (R) symbol shown in the flow charts of the logic of the device 50 indicates that a function at that location reads from or writes to a register. In addition, time values may be indicated throughout the logic flow charts, wherein a time value is surrounded by a square. These time values are indicated where certain decisions are time-sensitive and depend on the timekeeper in some embodiments. The times shown in the flow charts are estimated time values and are not intended to be limiting. These values are only provided as examples.

In certain embodiments that provide spoken instructions or messages as audio output include a printed circuit board with a simple microprocessor 24, in one embodiment. The microprocessor 24 may run the embedded software and interact with memory 26 in non-limiting embodiments. The embedded software may include a simple algorithm and keeps track of the sequence of scripts and the language. It retrieves, at the correct time, the proper script from a lookup table. In embodiments with error recognition, the embedded software is a recursive core algorithm and a number of subroutines. The algorithm may set register values based on events and uses register values to make decisions. The algorithm may further retrieve message scripts from a lookup table based on the value of register(s). The algorithm may handle interruptions which may come from buttons or sensors, in non-limiting embodiments.

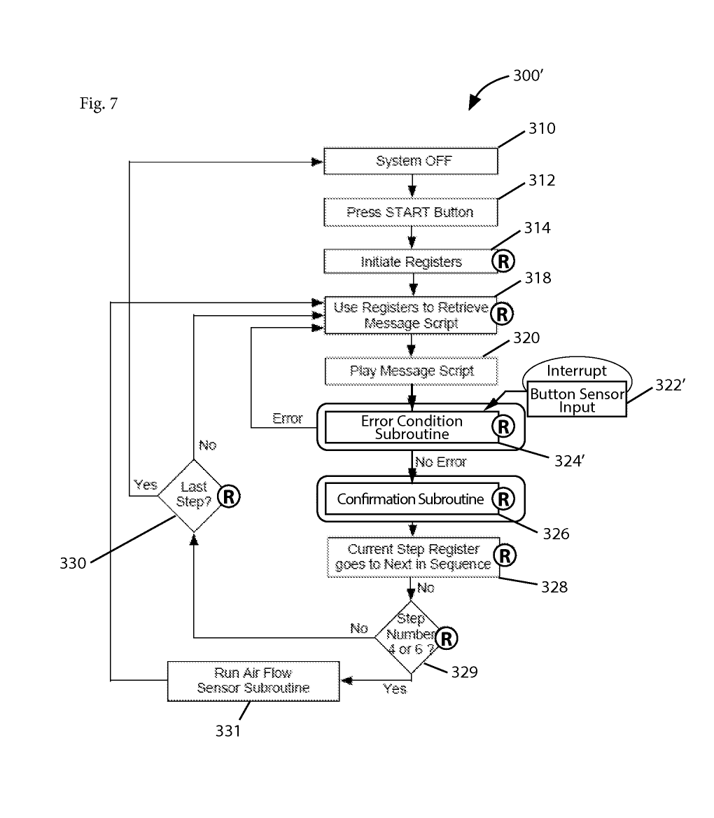

In regard to flow charts provided in FIGS. 7-10, several decisions are time-sensitive and depend on the timekeeping component. Therefore the time kept on the timekeeping component of the device 50 is designated at each time-sensitive location in the logic shown on the flowchart as a square with a specified time limit noted within. FIG. 7 provides the recursive core logic algorithm and flow chart for the training device.

In one embodiment of a respiratory inhaler device 50, particularly when the respiratory inhaler device 50 embodiment is used to train a user to use a metered dose inhaler medicament delivery inhaler device an inhalation flow rate of the user should be at least approximately 30 L/m. In an embodiment, the actuation mechanism 18′ should be activated once the inhalation flow rate reaches 30 L/m and the inhalation flow rate should continue at at least 30 L/m for at least two seconds after the actuation mechanism 18′ is activated to ensure that the medicament is received deep into the lungs of the user to receive a correct dose. The fluid flow rate sensor 20′ shown in FIG. 2 is used to determine the inhalation flow rate of the user.

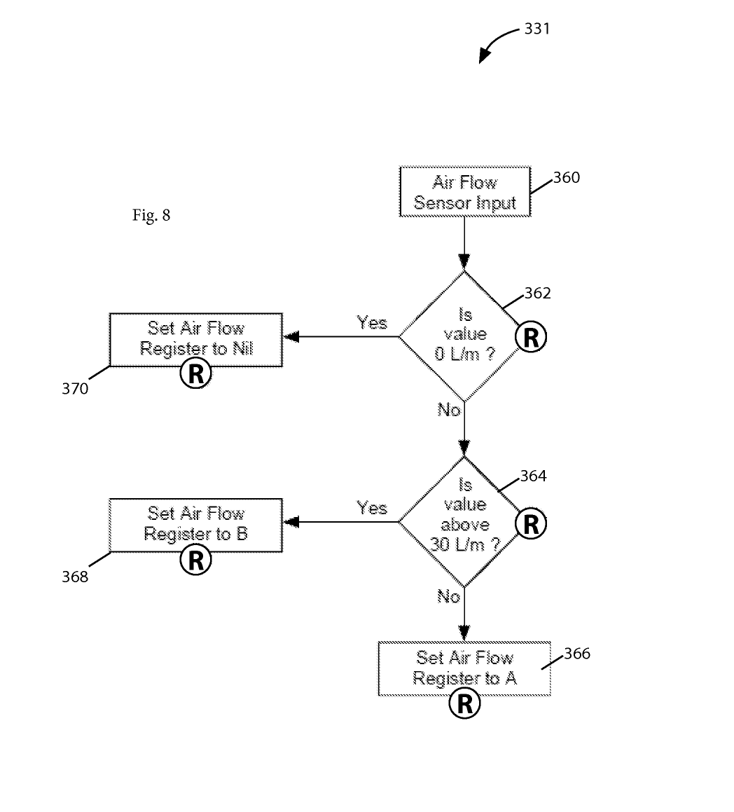

In FIG. 8, the fluid flow rate sensor 20′ (not shown) provides sensor input 360 to the air flow rate sensor subroutine 331. If the patient is not inhaling, the sensor measures 0 L/m in step 362 and sets the fluid flow register to Nil (default) 370. If the patient is inhaling between 0 L/m and 30 L/m in step 364, the fluid flow register is set to A in step 366, and if the patient is inhaling above 30 L/m the fluid flow (or air flow) register is set to B in step 368, as can be seen in the flow chart of FIG. 8.

The main algorithm as shown in FIG. 7 can be interrupted by actuation mechanism 18′ (which may include an actuation member or button) sensor input in step 322′, which may trigger an error correction subroutine 324′ shown in the flow chart of FIG. 9. This subroutine 324′ may evaluate whether the actuation mechanism sensor 322′ input represents an error condition. Each error condition has its own register value, in one embodiment. An error condition may start with a state transition of the actuation mechanism sensor from 0 to 1, in a non-limiting example. This state transition causes an interrupt in the main algorithm, in one embodiment.

In the flow chart of the embodiment of the logic of FIG. 9, the error condition subroutine 324′ can result in four outcomes: if there is no error condition, go to the “confirmation” subroutine 326 (described later). The error correction subroutine 324′ may determine that the actuation mechanism 18′ was activated too early in steps 372, and 376, and the current error register is set to X in step 380. If the subroutine 324′ determines that the inhalation was not long enough in steps 372 and 374, the current error register is set to Y in step 382, and if the 324′ determines that inhalation force (i.e., fluid flow rate) was not strong enough in steps 372 and 376, the current error register is set to Z in step 378.

As described above, the actuation mechanism 18′ should be activated (i.e., button is depressed, for example) after the fluid flow rate reaches 30 L/m, otherwise an error condition will result and current error register will be set to X step 380. Once the current error register is set, the logic goes to “use registers to retrieve message script” step 318 in the main algorithm 300′ of FIG. 7. Once the actuation mechanism 18′ is activated (i.e., actuation member is depressed in one embodiment), and no error has occurred, the user should continue to inhale for another 2.0 seconds. If the user fails to do so, an error condition will occur. If this error condition occurs, the current error register is set to Y in step 382 of error condition subroutine 324′, where after, the logic goes to “use registers to retrieve message script” step 318 in the main algorithm of FIG. 7.

If the fluid flow rate (air flow rate) is less than 30 liters/minute, the air flow will not carry the aerosol droplets deep into the lungs. After the actuation mechanism is activated, the patient should continue to inhale at a volumetric flow rate of 30 L/m for another 2.0 seconds. If the flow rate drops below 30 L/m during those 2.0 seconds, there is an error condition. If this error condition occurs, the current error register is set to Z as in step 378. Thereafter, the logic will proceed to the “Use Registers to Retrieve Message Script” step 318 in the main algorithm 300′ shown in FIG. 7.

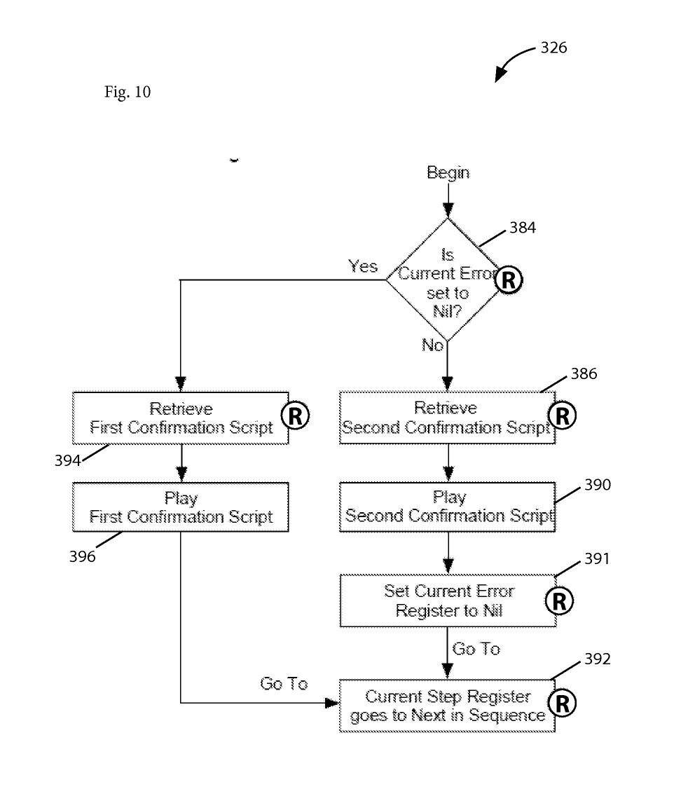

If the error condition subroutine 324′ determines that the user did not make an error, it proceeds to this confirmation subroutine 326 shown in the flow chart of FIG. 10. If an inhalation is above 30 L/m and lasts at least 2.0 seconds, then the device lets the user know that the inhalation was good, wherein a first confirmation script is retrieved in step 394, thereafter the first confirmation script is played in step 396.

If the user did not make a mistake, there are two situations that may occur as shown in the confirmation subroutine 326 of FIG. 10, the first of which is that the user did not make a mistake the previous time this same step was executed (current error register is Nil) as determined in step 384, thus, the user inhaled properly. The device 50 will play the message that the inhalation was good as in steps 394 and 396, and then goes on to “Current Step Register goes to Next in Sequence” of step 392 in the main algorithm 300′ of FIG. 7.

The second situation includes where the user did make a mistake in the previous attempt at this step (Current Error register is either X, Y, or Z). In that case, the device may provide positive reinforcement in the way of a confirmation message, in step 386 wherein the second confirmation script is retrieved, and step 390, wherein the second confirmation script is played. Following the execution of the step, the device will play a confirmation message to confirm that the step was completed correctly. Thereafter, the logic will set the Current Error register to Nil in step 391 and proceed to the “Current Step Register goes to Next in Sequence” step 392 function in the main algorithm 300′ of FIG. 7.

Errors occurring during use of the training device may be stored in a memory on or associated with the device. A history of the training record may be stored, including information such as number of errors, number of repeated errors, time between training sessions, number of training sessions, among other information, in non-limiting embodiments.

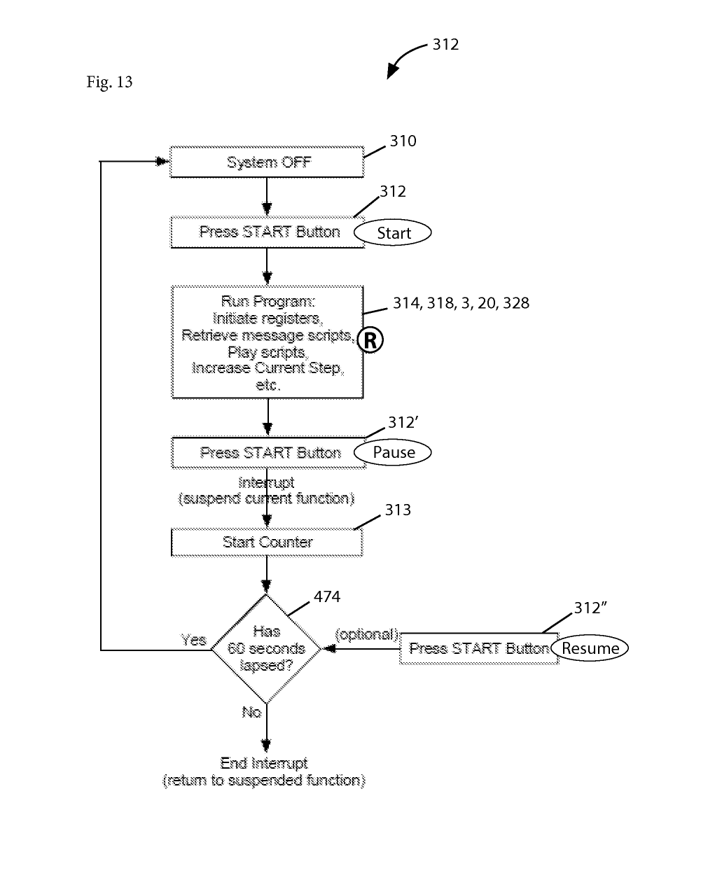

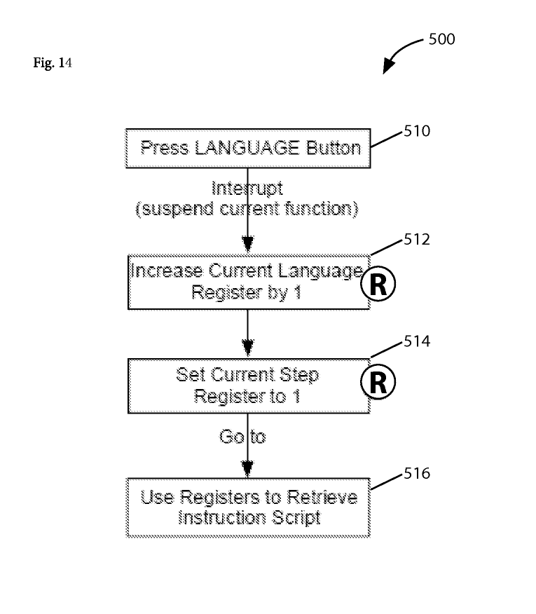

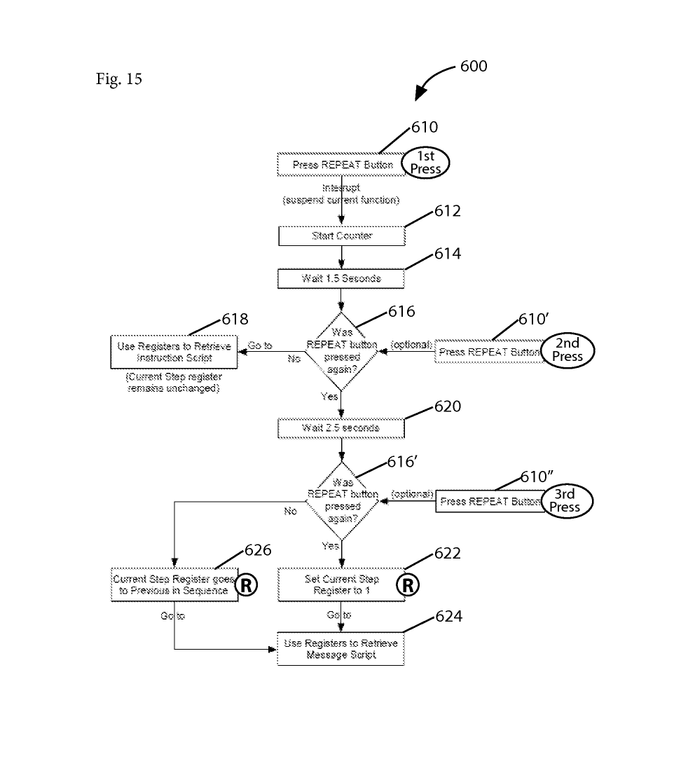

Embodiments of the respiratory inhaler training device 50, when used to train for a metered dose inhaler medicament delivery device may also include button subroutines to start and resume use of the device as shown in the flow chart of FIG. 13, and to change the language of the instructions of the training device as shown in the flow chart of FIG. 14, and furthermore subroutines for the use of the previous button as shown in the flow chart of FIG. 15.

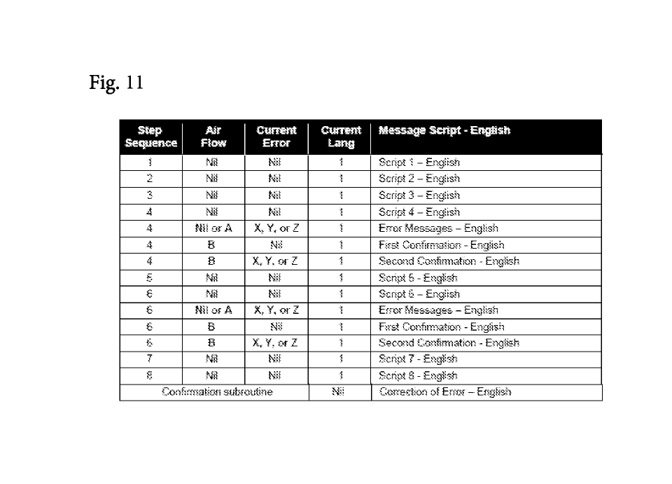

FIG. 11 provides an embodiment of a data structure of the system. The first three columns of the table are register values and the fourth column lists message scripts which may be provided as MP3 files in a non-limiting embodiment. The registers determine which MP3 file to retrieve and play, for example. The functions and subroutines that retrieve a message script retrieve the corresponding message script based on the current values of the three registers, in one embodiment.

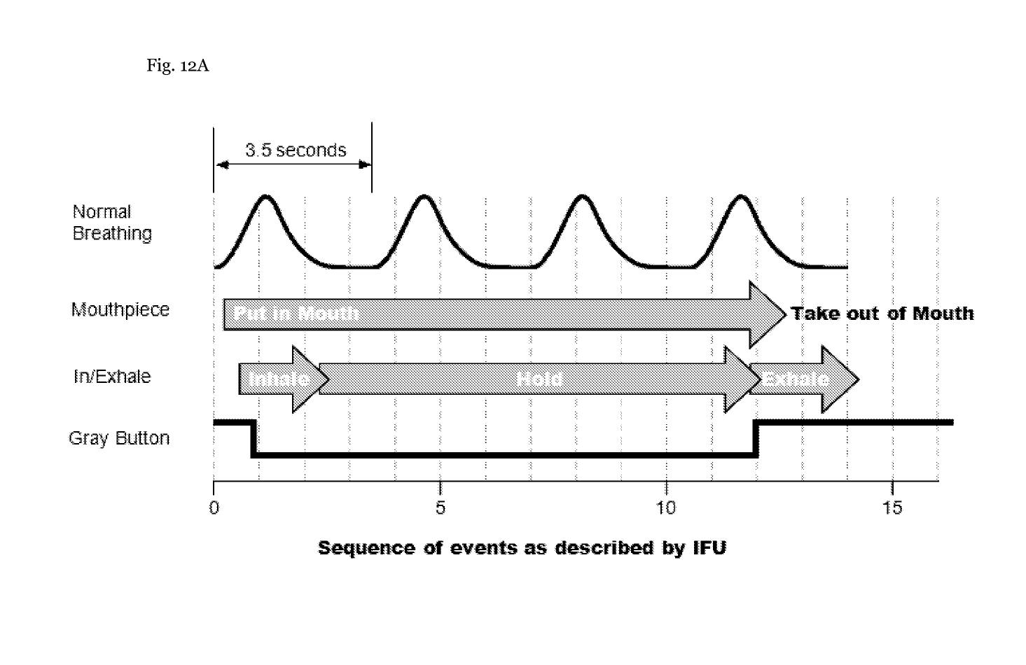

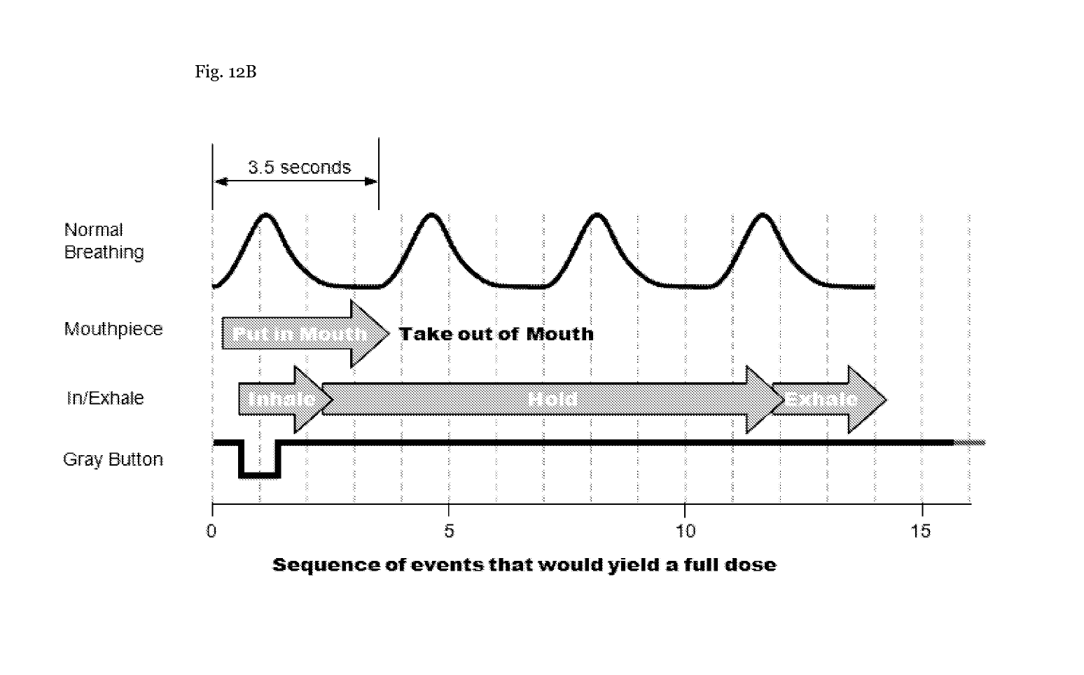

FIGS. 12A and 12B provide illustrations of two different examples of synchronized uses of the respiratory inhaler training device which would provide a correct dose of medicament to a patient using a metered dose inhaler medicament delivery device. In FIG. 12A, the inhalation begins prior to the activation of the actuation mechanism, and the actuation mechanism is activated while the user holds his or her breath and until the user begins to exhale, resulting in a complete dose of medicament and a correct usage of the device. In FIG. 12B, a user begins to inhale and activates the actuation mechanism at the same time, wherein the actuation mechanism is released (inactivated) during the remainder of the inhalation period of the user, and while the user subsequently holds his or her breath to receive the medicament deep into the lungs. This use of the metered dose inhaler device will also result in a complete dose of medicament, and consequently, a correct use of the respiratory inhaler training device.

A non-limiting example of the instructions for use (i.e., stepwise instructions) for the metered dose inhaler training device are as follows:

1. Hello. Welcome to the training Inhaler practice session. (Pause)
The training inhaler contains no medication. (Pause)

This practice session will help teach you how to use your training device. For complete instructions, make sure you read the full Patient Information provided with your prescription of medication. (Pause)

2. the training device consists of the following parts: the inhaler; the mouthpiece cover; the mouthpiece, the medicament canister or training canister; and the actuation counter located at the top of the canister. (Pause)
3. We are going to get started by walking you through the steps to practice using the training Inhaler. (Pause)
Shake the Inhaler well for 5 seconds before each use and remove the mouthpiece cover. Now, let's give it a try. (Pause)
4. First, breathe out fully and place the mouthpiece in your mouth and close your lips around it. Make sure that the inhaler is upright and that the opening of the mouthpiece is pointing towards the back of your throat. (Pause)
Start breathing in slowly and deeply through your mouth and immediately press down firmly on the top of the inhaler^{C}. Continue to breathe in and hold your breath for about 10 seconds so the medicine reaches your lungs.^{A,B} (Pause)
Now, remove the inhaler from your mouth. (Pause)
[If there was no error detected:] Great job!¹ or You successfully corrected the error! Great job!² (Pause)
5. For the second dose, shake the Training Inhaler again for 5 seconds. (Pause)
[Step 5 may optionally be provided in non-limiting embodiments. In some embodiments, the instructions will continue from step 4 above to step 6 below.]
6. Let's repeat the steps. (Pause)
First, breathe out fully and place the mouthpiece in your mouth and close your lips around it. Make sure that the inhaler is upright and that the opening of the mouthpiece is pointing towards the back of your throat. (Pause)
Start breathing in slowly and deeply through your mouth and immediately press down firmly on the top of the inhaler^{C}. Continue to breath in and hold your breath for about 10 seconds so the medicine reaches your lungs.^{A,B} (Pause)
Now, remove the inhaler from your mouth. (Pause)
[If there was no error detected:] Great job!¹ or You successfully corrected the error! Great job!² (Pause)
7. Congratulations! You have successfully completed your training session.

### (Pause)

8. Before taking your Symbicort dose, please read all the patient information with your prescription.

### Error Conditions (not Verbalized in the Symbicort Training Inhaler):

^{A} Inhalation is not long enough (2 seconds after depressing the button)
^{B} Inhalation force is not strong enough
^{C} Gray button is being depressed before breathing in

### Error Correction Scripts:

^{A} Beep or chime “We detected that you didn't inhale long enough. Let's try it again by breathing in longer.”
^{B} Beep or chime “We detected that your deep breath in wasn't strong enough. Let's try it again by breathing in slowly and deeply.”
^{C} Beep or chime “We detected that the top of the inhaler was pressed down before you began to breathe in. Let's try this again and remember to begin breathing in slowly and deeply before you press the top of the inhaler to release the medication.”

### Confirmation Scripts:

First Confirmation script: ¹Great job!
Second Confirmation script: ²You successfully corrected the error! Great job!

In a further embodiment, a respiratory inhaler training system may further include a signal receiving component for receiving the audible output from the signal output component. The signal receiving component may include a component of an external or remote device, and/or a component of the housing. As shown in the non-limiting embodiment of the respiratory inhaler training system 900 FIG. 4, an audible sound may be produced, for example by the actuation of the device 50, when the canister portion 19 is pressed into the housing of the device 50. This and other sounds native to the use of the device, for example, may be received by the signal receiving component 98 of the container 13′, in a non-limiting example, and may be processed by the components of the container 13′ (i.e., the microprocessor), wherein a feedback may be provided to the user based thereon. Alternatively or in addition, the user of the device may hear the audible output from the system, such as in the case of the audible output examples provided below. In some other non-limiting embodiments, the housing of the device may include multiple components which may produce mechanical sounds when they are moved relative to one another, wherein the sounds can be received by the signal receiving component 98 of the container, in a non-limiting embodiment, or by any other external or remote device, or a component of the housing of the device itself. Furthermore, these sounds may be heard by the user of the system and may be used to provide feedback to facilitate proper use of the system. In one embodiment, the signal receiving component may include a microphone. In one embodiment, the audible output may include a click, a whistle, or a mechanical sound producing component of the system, or the housing, or one or more components of the housing as described above, or furthermore, the canister moving relative to the housing, in non-limiting embodiments.

In yet a further embodiment, the respiratory inhaler training system includes a microprocessor, wherein the microprocessor includes a timekeeping component, the microprocessor being configured to receive and process a signal received from said signal receiving component, wherein the microprocessor detects correct and/or incorrect use of the system by a user. In still a further embodiment, the system includes a storage medium component associated with the microprocessor, wherein the microprocessor comprises a database of instructions pertaining to a sequence of steps for using the system, wherein said instructions are provided to a user via the signal output component. The system may be able to detect and provide feedback regarding errors and correct usage of the system based on the output from the system, received by the signal receiving component.

In a further embodiment, the respiratory inhaler training device or system described herein is provided wherein the time keeping component measures the elapsed time between a user's actions or events. In still a further embodiment, the time keeping component measures the elapsed time of a series of fluid flow rate values above a predetermined value.

In a further embodiment, the one or more predetermined fluid flow rate values comprise a value of at least 30 Liters per minute. In a further embodiment, the correct predetermined time includes but is not limited to a time between 0-2 seconds.

### Power Source

The amount of power available to supply the electronics is a challenge as space is often limited in the training devices. This requires including a high amount of energy density in a small space. For the trainer, considerations for viable battery technologies include primary disposable or secondary rechargeable batteries which may be removable and sealable inside the device.

Powering on the device, in some non-limiting embodiments, may initiate or activate the sequence of instructions from the device or container to the user. However, the instructions may be initiated or activated by any suitable means known in the art. For example, in another embodiment, activation of the actuation mechanism or removal of a protective cap or cover may initiate the sequence of instructions of the device. In yet another embodiment, the sequence of steps of instructions may be initiated by moving the device, which may be recognized via a motion sensor on or associated with the device. In still another embodiment, a user input via the responsive member of the device may activate or initiate the instructions.

As will be appreciated by one of skill in the art, certain examples of the present invention may be embodied as a device or system comprising a processing module, and/or computer program product comprising at least one program code module. Accordingly, the present invention may take the form of an entirely hardware embodiment or an embodiment combining software and hardware aspects, commonly known as firmware. As used herein, firmware comprises a computer program module that is embedded in a hardware device, for example a microprocessor or microcontroller. It can also be provided on flash memory or as a binary image file that can be uploaded onto existing hardware by a user. As its name suggests, firmware is somewhere between hardware and software. Like software, it is a computer program which is executed by a microprocessor or a microcontroller, but it is also tightly linked to a piece of hardware, and has little meaning outside of it in an embodiment.

Certain embodiments of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer-readable program code modules. These program code modules may be provided to a processing module of a general purpose computer, special purpose computer, embedded processor or other programmable data processing apparatus to produce a machine, such that the program code modules, which execute via the processing module of the computer or other programmable data processing apparatus, create means for implementing the functions specified in the flowchart and/or block diagram block or blocks.

Computer program code modules for carrying out the logic or operations of certain embodiments of the present invention may be written in an object oriented, procedural, and/or interpreted programming language including, but not limited to, Java, Smalltalk, Perl, Python, Ruby, Lisp, PHP, “C”, FORTRAN, Assembly, or C++. The program code modules may execute entirely on the device, partly on the device, as a stand-alone software package, partly on the training device and partly on a remote computer or device or entirely on the remote computer or device, the program code modules may execute entirely on the container, or partly on the device and partly on the container. In the latter scenario, the remote computer or device may be connected to the user's device through a local area network (LAN) or a wide area network (WAN), or the connection may be made to an external computer (for example, through the Internet using an Internet Service Provider).

One or more of the program code modules can include a records and statistical analysis feature, and can download and/or transfer records to and from the device. The program code modules may be helpful in research and development of the device. With the use of the program code modules recording and tracking various features and uses of the device, one can readily determine areas in which the device may be improved. The program code modules also include graphing capability of recorded data, as well as data trending results of the performance of the device and/or the user, the efficiency of the user and of the device in training and/or simulation. As part of the program code modules, features such as an output, for example, an alarm or indication (visual, auditory, tactile, or other sensory means) to the user of the device or to another can be initiated if the data received and analyzed by the module is out of range or is trending out of range (a range can be pre-determined).

What is claimed is:

1. A respiratory inhaler training device configured to provide stepwise instructions for using the device to a user in a sequence of steps, the respiratory inhaler training device comprising:
a housing defining a channel with an inlet and an outlet;
at least one actuation mechanism simulating provision of medicament;
at least one fluid flow rate sensor positioned so as to detect a rate of fluid flow through the channel to determine a fluid flow rate;
a signal output component for providing an output;
a microprocessor comprising a timekeeping component;
a storage medium component associated with the microprocessor comprising a database of instructions pertaining to the sequence and/or timing of steps for using the device stored thereon;
one or more program code modules stored on the microprocessor or the storage medium component, or a combination thereof, wherein the one or more program code modules comprise
a first program code module for causing the microprocessor to provide a first instruction;
a second program code module for causing the microprocessor to provide a subsequent instruction based on a current register; and
a third program code module causes the microprocessor to compare one or more fluid flow rate values detected with one or more predetermined fluid flow rate values, wherein when the detected fluid flow rate values do not meet the predetermined fluid flow rate values, an error condition is set in the current register, and a signal output component is initiated to provide an error message to the user.

2. The respiratory inhaler training device of claim 1, further comprising a fourth program code module, said fourth program code module causes the microprocessor to obtain the one or more fluid flow rate values over an elapsed time period, such that an error condition is set in the current register and a signal output component is initiated to provide an output to the user if the values obtained in the elapsed time period do not conform to predetermined values.

3. The respiratory inhaler training device of claim 1, further comprising at least one contact sensor.

4. The respiratory inhaler training device of claim 3, wherein the at least one contact sensor comprises an actuation sensor, said actuation sensor being disposed between the housing and the actuation mechanism said actuation sensor configured to provide a signal to the microprocessor when the actuation mechanism has been activated and/or inactivated.

5. The respiratory inhaler training device of claim 1, wherein the current register comprises information about a current step number, a current error condition and/or a current language.

6. The respiratory inhaler training device of claim 5, wherein the current step number is based on the sequence of steps, user actions, and/or sensor input.

7. The respiratory inhaler training device of claim 1, wherein the device further comprises at least one responsive member reactive to user input, and wherein the user input comprises a selection to return to a previous instruction, pause the stepwise instructions, move forward to the next instruction, power on or off the device, play the instruction script, and/or adjust the audio volume of the device.

8. The respiratory inhaler training device of claim 1, wherein the current register comprises information about a current step number and a current error condition.

9. The respiratory inhaler training device of claim 1, wherein based on an input received from the fluid flow rate sensor, the microprocessor detects whether an error condition has occurred, wherein when an error condition occurs, the microprocessor sets a current error in the current register.

10. The respiratory inhaler training device of claim 1, wherein following a current error set in the current register, correct completion of the step in which the error condition previously occurred removes the current error in the current register and resets the current register to zero.

11. The respiratory inhaler training device of claim 8, wherein when an error condition occurs, the subsequent instruction comprises a corrective instruction.

12. The respiratory inhaler training device of claim 8, wherein when an error condition has occurred, an error message is output to the user.

13. The respiratory inhaler training device of claim 1, wherein the signal output component comprises one or more speakers, and wherein error messages, confirmation messages, and/or corrective instructions are provided to the user by way of an audio output.

14. The respiratory inhaler training device of claim 1, wherein the signal output component comprises at least one or more visual stimuli such that the instructions are provided to the user by way of a visual output.

15. The respiratory inhaler training device of claim 1, wherein when a last instruction of the sequence of steps is executed, the device is powered off.

16. The respiratory inhaler training device of claim 1, wherein when an error condition occurs in a step in which the same error condition previously occurred a predetermined number of times, the device is powered off.

17. (canceled)

18. (canceled)

19. The respiratory inhaler training device of claim 1, wherein an error condition in the use of the device is detected based on the fluid flow rate sensor and/or a condition of the device relative to at least one predetermined value for the fluid flow rate sensor and/or the condition of the device as stored on the storage medium component.

20-27. (canceled)

28. The respiratory inhaler training device of claim 2, wherein the predetermined time period value is based on the fluid flow rate detected, the number of inhalations, and a predetermined volume of fluid required for each inhalation to receive a correct dose of a medicament.

29. The respiratory inhaler training device of claim 28, wherein a predetermined volume of fluid required for each inhalation to receive a correct dose of the medicament comprises between 0 and 2 liters.

30. The respiratory inhaler training device of claim 29, wherein a predetermined volume of fluid required for each inhalation to receive a correct dose of the medicament comprises between 0.5 and 1.5 liters.

31. The respiratory inhaler training device of claim 1, wherein the device further comprises an accelerometer to detect shaking of the device, wherein the timekeeping component record timestamps associated with shaking the device.

32. The respiratory inhaler training device of claim 31, wherein the timestamps are compared with the stepwise instruction in the sequence of steps for shaking the device, such that a signal output component is initiated in response to the comparison.

33. The respiratory inhaler training device of claim 32, wherein when the timestamps for shaking the device are recorded at a point in the stepwise instructions in which an instruction to shake the device is provided, an error message is output to the user and a current error is set in the current register.

34. The respiratory inhaler training device of claim 1, further comprising at least one orientation sensor.

35. The respiratory inhaler training device of claim 2, wherein when said actuation sensor is activated before the fluid flow rate sensor detects fluid flow in the channel, a current error is set in the current register, an error message is provided to the user and/or a corrective instruction is provided to the user.

36. The respiratory inhaler training device of claim 35, wherein T₀ comprises a time period until an inhalation begins as detected by the fluid flow rate sensor, T₁ comprises a time period between inhalation and activation of the actuation mechanism, and T₂ comprises a time period after activation of the actuation mechanism until the end of the inhalation, wherein when T₁ comprises a time between approximately 0-2 seconds, a confirmation message is output to the user.

37. The respiratory inhaler training device of claim 14, wherein the visual stimuli comprises an array of LED's, and/or a digital display.

38. A respiratory inhaler training system configured to provide instructions for using a respiratory inhaler training device to a user in a sequence of steps, said system comprising:
a respiratory inhaler training device comprising a housing defining a channel with an inlet and an outlet, said respiratory inhaler training device comprising an actuation mechanism, said actuation mechanism configured to simulate provision of medicament;
a respiratory inhaler training container, wherein said training device communicatingly connects to the respiratory inhaler training container;
a signal output component associated with the container and/or the training device, said signal output component configured to provide a feedback to the user based on a use of the system; and
a microprocessor associated with the respiratory inhaler training device and/or container configured so as to control a provision of the instructions to the user in the sequence of steps, wherein the respiratory inhaler training container comprises a power source.

39-42. (canceled)

43. A respiratory inhaler training system, comprising:
a housing defining a channel with an inlet and outlet, said housing comprising two or more mechanical components movable relative to one another during use of the system, wherein movement of the two or more mechanical components relative to one another produces an audible output;
a signal output component;
at least one actuation mechanism associated with the housing, said actuation mechanism simulating provision of medicament; and
a signal receiving component for receiving the audible output from the signal output component.

44-47. (canceled)

48. The respiratory inhaler training system of claim 43 wherein the audible output comprises a click, a whistle, or a mechanical sound producing component of the system.

49-89. (canceled)
